(19) United States
(12) Patent Application Publication
(10) Pub. No.: US 2007/0088382 A1
(43) Pub. Date: Apr. 19, 2007

(54) Embolic protection recovery catheter assembly

(76) Inventors: Nianjiong J. Bei, Foster City, CA (US); Earl A. Bright II, San Jose, CA (US); Andy E. Denison, Temecula, CA (US); Gregory W. Fung, San Mateo, CA (US); William J. Harrison, Signal Mountain, TN (US); Daniel S. Heilbrunn, Vancouver (CA); Danielle D. Laflash, San Mateo, CA (US); Jeffrey G. Lew, Murrieta, CA (US); Joanna Lubas, Fremont, CA (US); Paul F. Muller, San Carlos, CA (US); Alexander Nikanorov, Palo Alto, CA (US); John E. Papp, Temecula, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 11/249,723

(22) Filed: Oct. 13, 2005

(51) Int. Cl. A61M 29/00

(52) U.S. Cl. 606/200

(57) ABSTRACT

An embolic protection recovery catheter assembly configured for retrieval of medical devices from vasculature. The catheter includes a distal end portion adapted for effectively retrieving devices. In one aspect, the distal end portion is collapsible and in another aspect it is expandable. In other aspects, the catheter includes sub-structure supporting the distal tip or a tear-away tip configured to aid in the recovery process.

### BACKGROUND OF THE INVENTION

The present invention relates generally to recovery catheters for use in vasculature. More particularly, the present invention is directed towards recovery catheters for filtering devices and systems which can be used when an interventional procedure is being performed in a stenosed or occluded region of a blood vessel to capture embolic material that may be created and released into the bloodstream during the procedure.

Embolic filtering devices and systems are particularly useful when performing balloon angioplasty, stenting procedures, laser angioplasty or atherectomy in critical vessels, especially in vessels where the release of embolic debris into the bloodstream can occlude the flow of oxygenated blood to the brain or other vital organs, which can cause devastating consequences to the patient. In fact, the embolic protection devices and systems are useful with any vascular interventional procedure in which there is an embolic risk. Recovery catheters are essential to the successful retrieval of such protection systems and thus, to the success of the interventional procedure being performed.

A variety of non-surgical interventional procedures have been developed over the years for opening stenosed or occluded blood vessels in a patient caused by the build up of plaque or other substances on the wall of the blood vessel. Such procedures usually involve the percutaneous introduction of the interventional device into the lumen of the artery, usually through a catheter. In typical carotid PTA procedures, a guiding catheter or sheath is percutaneously introduced into the cardiovascular system of a patient through the femoral artery and advanced through the vasculature until the distal end of the guiding catheter is in the common carotid artery. A guide wire and a dilatation catheter having a balloon on the distal end are introduced through the guiding catheter with the guide wire sliding within the dilatation catheter. The guide wire is first advanced out of the guiding catheter into the patient's carotid vasculature and is directed across the arterial lesion. The dilatation catheter is subsequently advanced over the previously advanced guide wire until the dilatation balloon is properly positioned across the arterial lesion. Once in position across the lesion, the expandable balloon is inflated to a predetermined size with a radiopaque liquid at relatively high pressures to radially compress the atherosclerotic plaque of the lesion against the inside of the artery wall and thereby dilate the lumen of the artery. The balloon is then deflated to a small profile so that the dilatation catheter can be withdrawn from the patient's vasculature and the blood flow resumed through the dilated artery. As should be appreciated by those skilled in the art, while the above-described procedure is typical, it is not the only method used in angioplasty.

Another procedure is laser angioplasty which utilizes a laser to ablate the stenosis by super heating and vaporizing the deposited plaque. Atherectomy is yet another method of treating a stenosed blood vessel in which cutting blades are rotated to shave the deposited plaque from the arterial wall. A vacuum catheter is usually used to capture the shaved plaque or thrombus from the blood stream during this procedure.

In the procedures of the kind referenced above, abrupt reclosure may occur or restenosis of the artery may develop over time, which may require another angioplasty procedure, a surgical bypass operation, or some other method of repairing or strengthening the area. To reduce the likelihood of the occurrence of abrupt reclosure and to strengthen the area, a physician can implant an intravascular prosthesis for maintaining vascular patency, commonly known as a stent, inside the artery across the lesion. The stent is crimped tightly onto the balloon portion of the catheter and transported in its delivery diameter through the patient's vasculature. At the deployment site, the stent is expanded to a larger diameter, often by inflating the balloon portion of the catheter.

Prior art stents typically fall into two general categories of construction. The first type of stent is expandable upon application of a controlled force, as described above, through the inflation of the balloon portion of a dilatation catheter which, upon inflation of the balloon or other expansion means, expands the compressed stent to a larger diameter to be left in place within the artery at the target site. The second type of stent is a self-expanding stent formed from, for example, shape memory metals or super-elastic nickel-titanium (NiTi) alloys, which will automatically expand from a collapsed state when the stent is advanced out of the distal end of the delivery catheter into the body lumen. Such stents manufactured from expandable heat sensitive materials allow for phase transformations of the material to occur, resulting in the expansion and contraction of the stent.

The above non-surgical interventional procedures, when successful, avoid the necessity of major surgical operations. However, there is one common problem which can become associated with all of these non-surgical procedures, namely, the potential release of embolic debris into the bloodstream that can occlude distal vasculature and cause significant health problems to the patient. For example, during deployment of a stent, it is possible that the metal struts of the stent can cut into the stenosis and shear off pieces of plaque which become embolic debris that can travel downstream and lodge somewhere in the patient's vascular system. Pieces of plaque material can sometimes dislodge from the stenosis during a balloon angioplasty procedure and become released into the bloodstream. Additionally, while complete vaporization of plaque is the intended goal during a laser angioplasty procedure, quite often particles are not fully vaporized and thus enter the bloodstream. Likewise, not all of the embolic created during an atherectomy procedure may be drawn into the vacuum catheter and, as a result, enter the bloodstream as well.

When any of the above-described procedures are performed in arteries, the release of embolic into the circulatory system can be extremely dangerous and sometimes fatal to the patient. Debris that is carried by the bloodstream to distal vessels of the brain can for example cause these cerebral vessels to occlude, resulting in a stroke, and in some cases, death. Therefore, although cerebral percutaneous transluminal angioplasty has been performed in the past, the number of procedures performed has been limited due to the justifiable fear of causing an embolic stroke should embolic debris enter the bloodstream and block vital downstream blood passages.

Medical devices have been developed to attempt to deal with the problem created when debris or fragments enter the circulatory system following vessel treatment utilizing any one of the above-identified procedures. One approach which has been attempted is the cutting of any debris into minute sizes which pose little chance of becoming occluded in major vessels within the patient's vasculature. However, it is often difficult to control the size of the fragments which are formed, and the potential risk of vessel occlusion still exists, making such a procedure in the carotid arteries a high-risk proposition.

Other techniques which have been developed to address the problem of removing embolic debris include the use of catheters with a vacuum source which provides temporary suction to remove embolic debris from the bloodstream. However, as mentioned above, there have been complications with such systems since the vacuum catheter may not always remove all of the embolic material from the bloodstream, and a powerful suction could cause problems to the patient's vasculature. Other techniques which have had some success include the placement of a filter or trap downstream from the treatment site to capture embolic debris before it reaches the smaller blood vessels downstream. However, there have been problems associated with filtering systems, particularly during the expansion and collapsing of the filter within the body vessel. If the filtering device does not have a suitable mechanism for closing the filter, there is a possibility that trapped embolic debris can backflow through the inlet opening of the filter and enter the blood-stream as the filtering system is being collapsed and removed from the patient. In such a case, the act of collapsing the filter device may actually squeeze trapped embolic material through the opening of the filter and into the bloodstream.

Certain of the available filters which can be expanded within a blood vessel are attached to the distal end of a guide wire or guide wire-like tubing which allows the filtering device to be placed in the patient's vasculature when the guide wire is manipulated in place. Once the guide wire is in proper position in the vasculature, the embolic filter can be deployed within the vessel to capture embolic debris. The next step then involves removing the captured debris and filter device from vasculature.

Since the efficient and effective retrieval of a filter which has captured vasculature debris can be highly critical to the success of an interventional procedure, the structure of a retrieval catheter must facilitate such retrieval. Accurately positioning a recovery catheter with respect to a filter or embolic protection device can be a concern especially where the interventional site is defined by tortuous or narrow anatomy. Moreover, the ability to manipulate a recovery catheter at the interventional site is highly critical as is the act of collecting a filter or embolic protection device. Accordingly, what is needed is a recovery catheter that embodies structural characteristics specifically designed to facilitate the accurate placement and manipulation of the recovery catheter at an interventional site. It is also desirable that a superior end portion of the recovery catheter be configured to facilitate the successful collection of both the filter/embolic protection devices and the debris collected thereby.

The present invention satisfies these and other needs.

### SUMMARY OF THE INVENTION

Briefly and in general terms, the present invention is directed towards a recovery catheter for use in vasculature. The recovery catheter can be employed to receive and retrieve various medical devices placed within vasculature of a patient.

In one aspect, the recovery catheter of the present invention is intended to be used to facilitate the retrieval of a filter or embolic protection device. The recovery catheter is thus equipped with a superior end portion configured to accept at least a portion of a filter or embolic protective device.

The recovery catheter has an elongate profile and a length sufficient to extend from an exterior of a patient to an interventional site within the patient's vasculature. An inferior end portion is designed to be manipulated by a physician or operator during advancement to the interventional site as well as once the site has been accessed.

In one particular aspect, the recovery catheter includes a superior end portion specifically suited for accommodating receiving a medical device. In a first embodiment, the superior end portion is collapsible about a medical device and folds as the medical device is withdrawn. Structure that provides a positive outward force on the superior end portion can be incorporated into the system to provide desired transitions in axial flexibility. The superior end portion can additionally include a tip made from radiopaque material and which embodies a section that tears as a filter is withdrawn into the recovery sheath. In a second embodiment, the superior end portion is expandable as the medical device is withdrawn.

In further aspects, the recovery catheter can include sub-structures providing torqueability within vasculature. Such a catheter is also contemplated to embody a superior end portion having a pre-shaped bend.

In still further aspects, a recovery catheter includes a reinforced radiopaque stopper intended to engage a recovery filter and aid in selectively positioning the filter within a catheter. The various catheters of the present invention can also be equipped with a swaged marker and overlapping tip assembly. Additionally, the superior end portion of the recovery catheter can embody a tip having a curve or bend.

Other features and advantages of the present invention will become apparent from the following detailed description, taken in conjunction with the accompanying drawings, which illustrate, by way of example, the principles of the invention.

### BRIEF DESCRIPTION OF THE DRAWINGS

FIG. 1 is a partial cross-sectional view, depicting a recovery catheter assembly having a collapsible tip;

FIG. 2 is a partial cross-sectional view, depicting the recovery catheter assembly of FIG. 1 with a filter device withdrawn within the collapsible tip;

FIG. 3 is a partial cross-sectional view, depicting a recovery catheter including a positive pressure sub-structure cooperating with a distal tip;

FIG. 4 is a partial cross-sectional view, depicting a recovery catheter including a distal end portion with a tear-away tip;

FIG. 5 is a partial cross-sectional view, depicting a catheter assembly with an expandable tip;

FIG. 6 is a partial cross-sectional view, depicting the catheter assembly of FIG. 5 with a filter device withdrawn within the expandable tip;

FIG. 7 is a partial cross-sectional view, depicting a recovery catheter equipped with a reinforced radiopaque stopper; and

FIG. 8 is a partial cross-sectional view, depicting a catheter tip assembly including a swaged marker and overlapping catheter tip.

### DETAILED DESCRIPTION OF THE INVENTION

Referring to the drawings, which are provided for example and not by way of limitation, there is shown a recovery catheter for use with a filter or embolic protection device. The recovery catheter of the present invention embodies structural characteristics specifically designed to effect the successful retrieval from vasculature of a filter or embolic protection device. The recovery catheter can include structure providing the device with a desirable longitudinal flexibility. Although the present invention is described for use with filters and embolic protection devices, it is to be recognized that the present invention can be used in conjunction with any medical device placed in vasculature or other areas of the body.

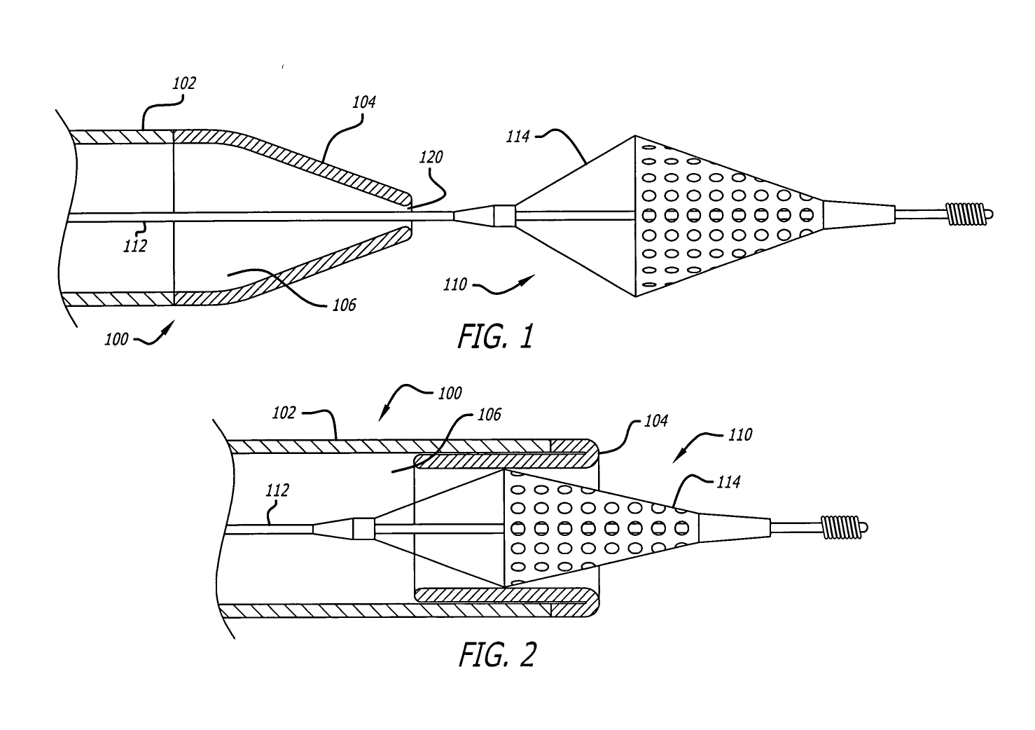

Turning now to FIGS. 1 and 2, there is shown one embodiment of a recovery catheter 100 of the present invention. The recovery catheter is elongate in shape and includes a generally tubular proximal portion 102 and a distal end portion 104 extending from the proximal portion 102. The proximal portion 102 is contemplated to have a length sufficient to extend from an outside of a patient's body to an interventional site within the patient. An internal bore 106 extends through the proximal portion 102 and distal end portion 104. The internal bore 106 is sized to receive a medical device such as a filter or embolic protection device 110.

The medical device 110 intended to be received by the recovery catheter 100 can assume various forms. In one form, the medical device 110 can include a longitudinally extending wire 112 connected to a basket or receptacle 114. As with the proximal portion 102 of the recovery catheter 100, the wire 112 is contemplated to have a length sufficient to extend from an exterior of a patient's body to the interventional site.

The distal portion 104 of the recovery catheter 100 is specially designed to receive a medical device 110. When used to recover a filter device, the distal end portion 104 defines a distally extending cone-like shape with an expandable opening 120 formed at an apex of the cone. When unexpanded, the opening 120 is sized to receive the wire 112 attached to the filter device, for example. In this way, the opening 120 can aid in centering the filter within vasculature. It is to be recognized that various other profiles for the distal end portion are also contemplated. Moreover, the opening can be offset, so that the wire 112 of the filter 100 or other device is offset in vasculature or other conduit or body organ.

The material of the distal end portion 104 is selected so that as the medical device 110 is longitudinally withdrawn within the recovery catheter 100, the distal end portion 104 folds internally upon itself. Such action aids in the effective and controlled retrieval of a medical device within the recovery catheter 100.

In a further aspect, a system involving the recovery catheter 100 can additionally include structure providing the catheter with a desired longitudinal or axial flexibility in the region of the distal end portion 104. This structure is intended to be modified during use such as by longitudinally moving the structure with respect to the distal end portion 104 or by varying the positive outward force provided thereby.

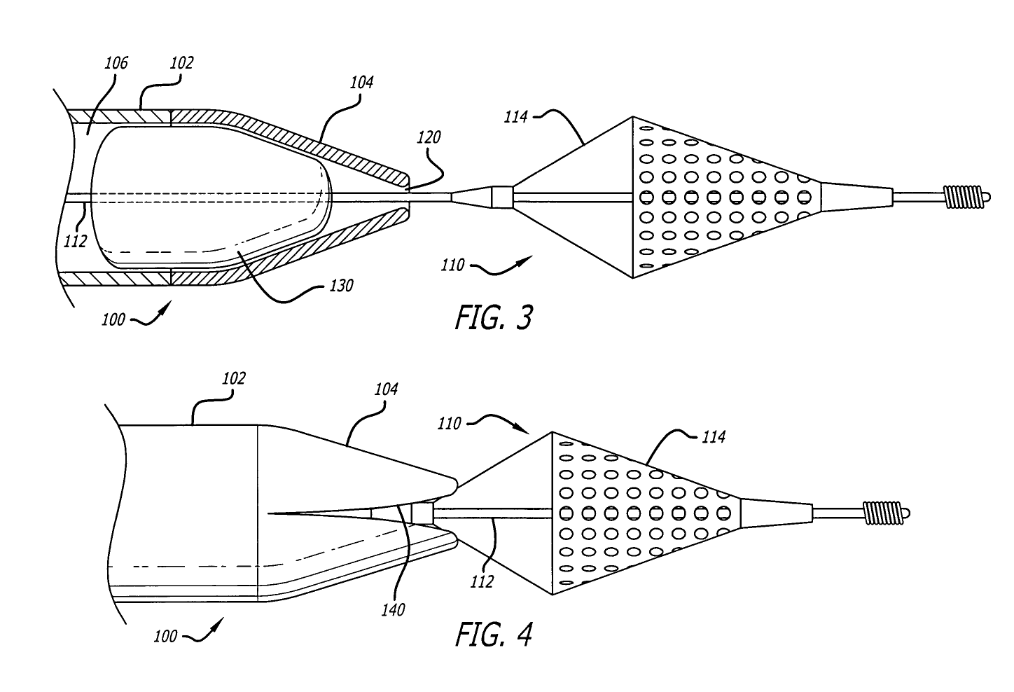

In one embodiment (FIG. 3), such variable structure is provided by an inflatable member 130. The inflatable member 130 can form a superior end portion of a catheter that is threaded over the wire 112 of the filter device 110 or can be formed as part of the assembly defining the filter device 110. The positive pressure structure 130 can also form part of the internal bore 106 of the recovery catheter 100. In any case, in the embodiment shown, varying degrees of fluid pressure is provided to the inflation member 130 so that is can exert a desired outward pressure against the internal bore of the recovery catheter 100 near the transition between the proximal portion 102 and the distal end portion 104 of the catheter.

In yet another aspect, as shown in FIG. 4, the distal end portion 104 of the recovery catheter 100 can be equipped with a seam 140. The seam 140 can extend varying lengths of the distal end portion and is intended to split as a medical device 110 is withdrawn within the recovery catheter 100. The distal end portion 104 can define a cone or other shape and can be designed to fold upon itself or simply open in response to the longitudinal mount of the medical device 110.

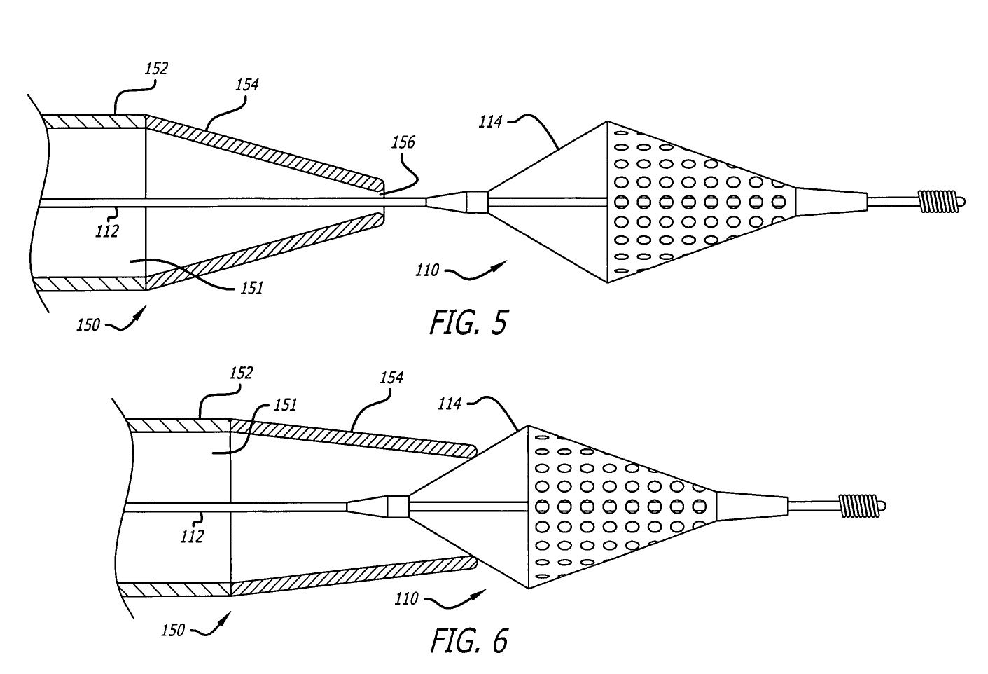

With reference now to FIGS. 5 and 6, there is shown an alternate embodiment of a recovery catheter 150. As before, this embodiment of a recovery catheter 150 is suited for retrieving or accepting various medical devices placed within a patient's body. In one particular use, the recovery catheter 150 is configured to accept the filter or embolic protection device 110.

The recovery catheter 150 is generally elongate and defines a tubular member having an internal bore 151. The catheter includes a proximal portion 152 and a distal end portion 154 connected to the proximal portion 152. The proximal end portion 152 has a length sufficient to extend from outside of a patient to an interventional site within a patient. In one aspect, the internal bore 151 is sized to receive both the basket or collection receptacle 114 and wire 112 of the medical device.

In a relaxed configuration, the distal end portion 154 has a generally conical shape. However, various other profiles can be employed as well. An opening 156 is configured in an apex or terminal end of the distal end portion 154. The opening 156 can be located to facilitate centering a medical device within a vessel, lumen or body catheter or can be off-centered for a particular application.

Moreover, as can be best seen in FIG. 6, the distal end portion can be made from flexible material so that it can be expanded into an open configuration. As the medical device 110 is withdrawn through the opening 156 and within the bore 151, the flexible distal end portion 154 opens about the medical device 114. Once the medical device 110 is pulled or otherwise placed completely within the bore 151, the distal end portion reassumes its relaxed, generally closed configuration. In other embodiments, however, the distal end portion can be made from material which is irreversibly deformed.

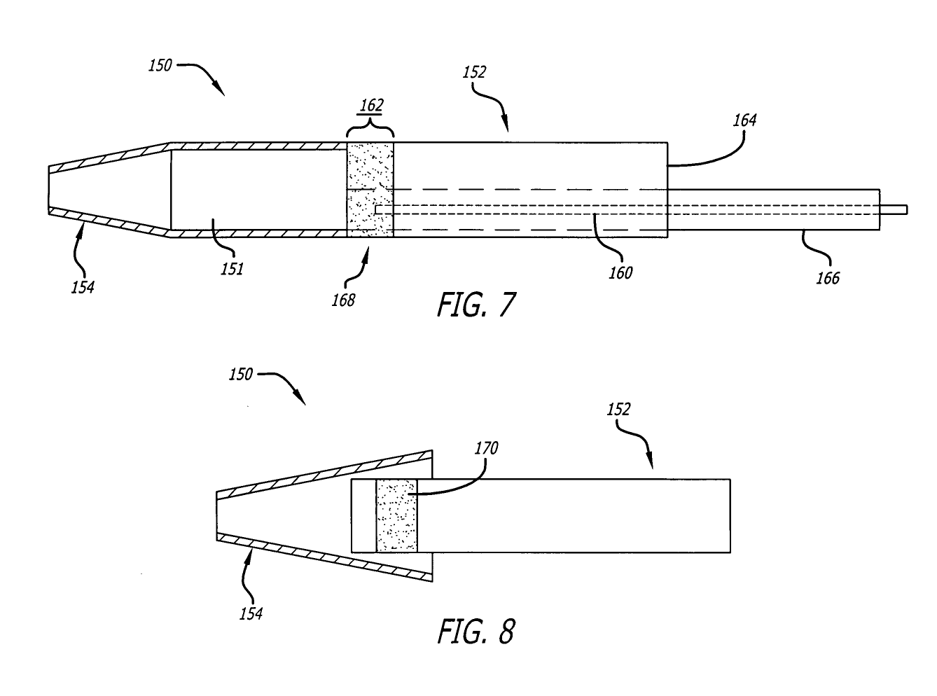

In each of the disclosed embodiments, a superior portion of the recovery catheter can be curved, angled or otherwise bent. Such a configuration is intended to be used to access vasculature or body portions which mimic such curves or bends or which are at some angle with respect to an approach pathway. The recovery catheter may further include substructure such as a longitudinally moveable mandrel 160 (See FIG. 7) for straightening the angled or curved superior end portion. Additionally, one or more wires can be affixed to the superior end portion to accomplish angling or bending the superior end portion with respect to more inferior portions of the recovery catheter.

In other aspects, the mandrel 160 can be affixed longitudinally with respect to the proximal portion 152. Whether the mandrel is independently moveable or not, a superior terminal end thereof can be equipped with a stop 162 intended to aid in locating the medical device within the bore 151 of the catheter 150. The stop 162 is intended to be made from radiopaque material to facilitate tracking the recovery of a medical device via remote viewing techniques. The stopper 162 is intended to prevent retraction of medical device 110 from exceeding the position of the stopper 162. The assembly also includes a rapid exchange port 164 through which the wire of a medical device can be threaded. A proximally extended tube 166 provides a space for the mandrel 160 or other wires (not shown) for steering the superior 168 end portion of the recovery catheter.

In yet other embodiments as shown in FIG. 8, the recovery catheter 150 can include a radiopaque marker 170 swaged on a terminal end portion of the proximal portion 152 of a recovery catheter. The distal end portion 154 is fixedly placed over the swaged radiopaque marker 170 to define the top of the recovery catheter 150. Such an approach results in covering any sharp edges of the radiopaque marker as well as aids in the fixation thereof to the recovery catheter 150.

Accordingly, the present invention is directed towards a recovery catheter embodying structural characteristics designed to effect the successful recovery of medical devices placed within a patient. In specific embodiments, the present invention is employed to recover filters or other embolic protection devices containing captured material from vasculature. However, as noted, the present invention can be used in conjunction with any medical device and furthermore, the various disclosed details and aspects of the present invention can be applied to each of the contemplated embodiments to create a device having characteristics which are desirable for a particular application.

Thus, it will be apparent from the foregoing that, while particular forms of the invention have been illustrated and described, various modifications can be made without parting from the spirit and scope of the invention.

1. A system for use in vasculature, comprising:
an elongated member;
a filter device connected to the elongated member; and
a recovery catheter, the recovery catheter including a proximal portion and distal end portion, wherein the distal end portion is flexible and capable of forming a cone-like shape in a first configuration and an inverted sleeve in a second configuration.

2. The system of claim 1, wherein the recovery catheter is longitudinally adjustable with respect to the filter device.

3. The system of claim 1, wherein the recovery catheter has a bore sized to receive the filter device.

4. The system of claim 1, wherein the distal end portion is collapsible about the filter device.

5. The system of claim 1, wherein the distal end portion folds inwardly about the filter device.

6. The system of claim 1, further comprising a positive pressure device cooperating with the distal end portion.

7. The system of claim 6, wherein the positive pressure device comprises an expandable balloon.

8. The system of claim 6, wherein the positive pressure device is positioned within a bore defined by the recovery catheter adjacent the distal end portion.

9. The system of claim 6, wherein the positive pressure device is affixed to the elongate member.

10. The system of claim 1, wherein the distal end portion includes a tear-away suit configured longitudinally along the distal end portion.

11. The system of claim 1, further comprising an affixed or moveable support mandrel configured within a bore of a the recovery catheter and a stopper affixed to a superior end of the mandrel.

12. The system of claim 1, wherein the distal end portion is configured about a superior end of the proximal portion, the superior end of the proximal portion including a marker swaged thereon.

13. A system for use in vasculature, comprising:
an elongated member;
a filter device connected to the elongated member; and
a recovery catheter, the recovery catheter including a proximal portion and a distal end portion, wherein the distal end portion is flexible and capable of forming a cone-like shape in a first configuration and an expanded sleeve in a second configuration.

14. The system of claim 13, wherein the recovery catheter is longitudinally adjustable with respect to the filter device.

15. The system of claim 1, wherein the recovery catheter has a bore sized to receive the filter device.

16. The system of claim 13, wherein the distal end portion is expandable about the filter device.

17. The system of claim 13, wherein the distal end portion opens about a filter device.

18. The system of claim 13, further comprising a positive pressure device cooperating with the distal end portion.

19. The system of claim 18, wherein the positive pressure device comprises an expandable balloon.

20. The system of claim 18, wherein the positive pressure device is positioned within a bore defined by the recovery catheter adjacent the distal end portion.

21. The system of claim 18, wherein the positive pressure device is affixed to the elongate member.

22. The system of claim 13, wherein the distal end portion includes a tear-away suit configured longitudinally along the distal end portion.

23. The system of claim 13, further comprising a support mandrel configured within a bore of a the recovery catheter and a stopper affixed to a superior end of the mandrel.

24. The system of claim 13, wherein the distal end portion is configured about a superior end of the proximal portion, the superior end of the proximal portion including a marker swaged thereon.

25. The system of claim 12, wherein the portion has a longitudinal axis, the distal end portion extending laterally with respect to the longitudinal axis.
